(19) United States
(12) Patent Application Publication
(10) Pub. No.: US 2005/0277184 A1
(43) Pub. Date: Dec. 15, 2005

(54) Stirring system for cell culture

(76) Inventor: Adrian Neil Bargh, London (GB)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 11/146,160

(22) Filed: Jun. 7, 2005

(30) Foreign Application Priority Data
Jun. 7, 2004 (EP) 04253376.0

(51) Int. Cl. C12M 1/02

(52) U.S. Cl. 435/286.7; 435/303.3; 435/305.2

(57) ABSTRACT

A stirring system (1) is disclosed for use in a cell culture system which handles a large number of cell culture vessel assemblies (2). Each cell culture vessel assembly includes a platen (23) on which are disposed a plurality of stirring rods (25), and which is transversely movable across the top of the respective culture vessel to provide for stirring of one or more samples within the culture vessel. The stirring system includes a support (10) configured to receive plural cell culture vessel assemblies at predefined positions around an axis (z) and has a drive rotor (14) disposed adjacent the support, the drive rotor being mounted for eccentric movement about the axis (z) of the support (10) with respect to the support. The drive rotor has plural of drive connections (24) for selectively engaging respective platens (23) of culture vessel assemblies supported at the predefined positions so as to enable simultaneous stirring of samples in culture vessels whose platens are engaged with the respective drive connections. A drive

[symbol]

### BACKGROUND

The invention relates to a system for stirring or shaking vessels containing cells which are processed in a cell culture system.

Protein production is an essential first step in many drug discovery processes. For example, E. coli and baculovirus are used routinely in the genomics biotechnology and pharmaceutical industries for protein production because their culture is simple and relatively rapid and, additionally, sufficient quantities of purified protein can be produced for screening within a few days at a relatively economic cost.

A system to automate protein production has been developed using micro-organisms and insect cells and which uses one or more incubators to enable cell growth to take place under controlled conditions. One requirement of an incubator is for aeration and agitation of the contents of vessels containing cells disposed in a suitable medium and the present invention is aimed at providing a suitable system capable of carrying out stirring and/or shaking operations.

### SUMMARY OF THE INVENTION

According to a first aspect of the present invention there is provided a stirring system for a cell culture system arranged to handle a plurality of cell culture vessel assemblies each of which includes a platen on which are disposed a plurality of stirring rods, and which is transversely movable across the top of the respective culture vessel to provide for stirring of one or more samples within the culture vessel, the stirring system including:
a support configured to receive plural cell culture vessel assemblies at predefined positions around an axis;
a drive rotor disposed adjacent the support, the drive rotor being mounted for eccentric movement about the axis of the support with respect to the support and having a plurality of drive connections for selectively engaging respective platens of culture vessel assemblies supported at the predefined positions to enable simultaneous stirring of samples in the plural culture vessels whose platens are engaged with the respective drive connections; and
a drive transmission system for connecting the drive rotor to a drive motor.

Such a system enables all the attached culture vessel assemblies to be stirred by a single drive system, avoiding the need for plural drives, ie individual drives for each culture vessel assembly within an incubator.

The cell culture assemblies are preferably of the type described in our European Patent Application no. 03255896.7.

A further aspect of the invention includes a stirring system for a cell culture system arranged to handle a plurality of cell culture vessel assemblies, the stirring system including:
a support configured to receive plural cell culture vessel assemblies at a plurality of predefined positions around an axis;
a drive rotor disposed adjacent the support, the drive rotor having a corresponding plurality of sets of stirring rods for disposing in use in respective culture vessel assemblies supported at the predefined positions on the support, and being mounted for eccentric movement about the axis of the support with respect to the support to vibrate the stirring rods to enable stirring of samples in the plural culture vessels simultaneously; and
a drive transmission system for connecting the drive rotor to a drive motor.

The support may be rotatable around the axis to move the cell culture vessel assemblies between a number of positions about the axis. The drive rotor may be disposed on the support.

The drive transmission system preferably includes a plurality of eccentric bearings disposed on one or both of the rotor and the support and may also include a drive pulley mounted about the axis and a drive belt disposed around the drive pulley, and each of the bearings includes a pulley around which the drive belt is disposed to drive the rotor.

Each of the drive connections preferably includes a magnetic coupling engaging a corresponding magnetic coupling on a respective culture vessel assembly movable platen. Similarly, the culture vessel assemblies may be magnetically coupled to the support.

The culture vessel assemblies are advantageously each coupled to the support by means of a socket on the support arranged to receive a corresponding plug-in portion of the culture vessel assembly.

One or more pneumatic actuators may be disposed and actuatable to disengage culture vessel assemblies from the support.

Channels are preferably provided in the support for supplying gas or liquid to the culture vessel assemblies in use. The stirring rods are also preferably solid, but alternatively may be tubular for example for connection to the channels in the culture vessel assemblies for supplying gas or liquid into the samples in the culture vessel assemblies.

The support may be configured to receive cell culture vessels in two or more rings substantially coaxial with the axis.

The invention also includes a cell culture system including an incubator having a housing and, disposed within the housing a stirring system as defined above. A plurality of stirring systems can be disposed within the incubator housing one above another.

### BRIEF DESCRIPTION OF THE DRAWINGS

Example of systems according to the present invention will now be described with reference to the accompanying drawings in which:—

FIG. 1 illustrates in perspective from above, a stirring system according to the invention for use in an incubator;

FIG. 2 illustrates the stirring system in another perspective view from one side; and

FIG. 3 is an perspective view of a culture vessel assembly for use with the system shown in FIGS. 1 and 2.

### DETAILED DESCIPTION

The system shown in the drawings is for use within an incubator which includes a housing in order to enable a controlled environment to be maintained and adjusted. For simplicity the housing of the incubator is not shown in the drawings as its shape and size relative to the stirring system is not material to the invention.

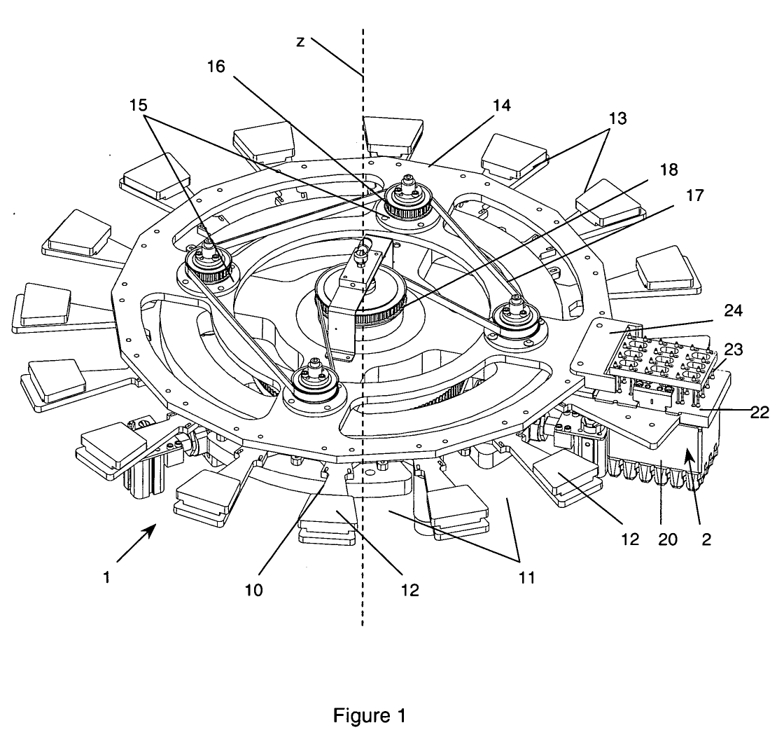

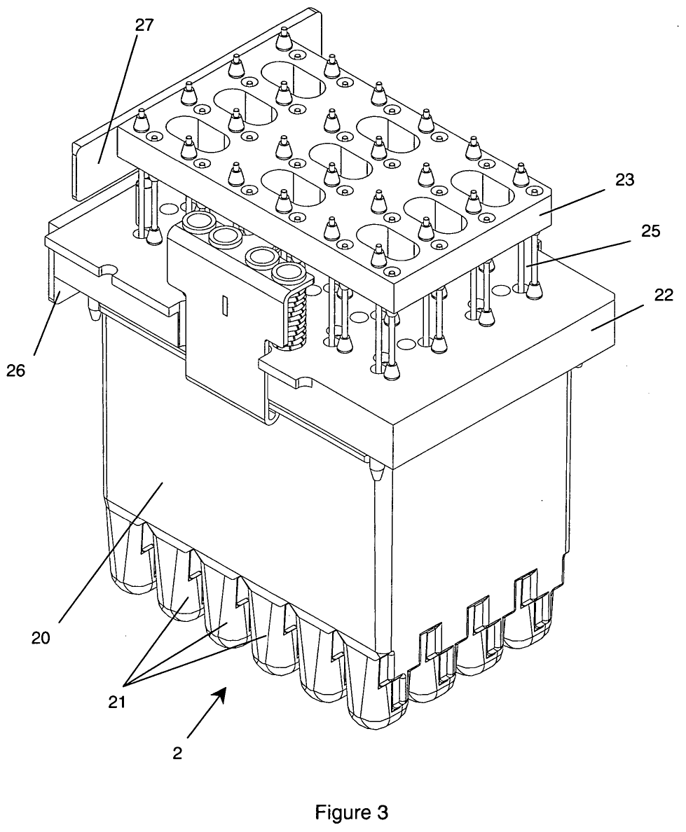

The stirring system 1 includes a rotatable support 10 in the form of a rotatable disc defining, around its periphery, a series of support locations 11 by means of protruding, generally radial, flanges 12 which define substantially parallel sides 13 for the sockets defining the support locations 11. The support is rotatable about an upright axis z. In an alternative construction (not shown) the support is fixed in position, ie non-rotatable. On the right hand side of FIG. 1 there is shown a culture vessel assembly 2 of the type generally described in our European patent application no. 03255896.7, and shown in more detail in FIG. 3, which has a culture vessel block 20 with multiple wells 21 for containing cells suspended or supported in a suitable medium.

The culture vessel block 20 has a detachable lid or platen 22 on which is mounted, spaced apart from the lid or platen 22, a plate 23 forming a movable platen which, as described in our European patent application no. 03255896.7, supports a plurality of stirring rods 25 extending downwardly from the platen 23, through the lid (platen) 22, and into the individual wells 21. The platen forms a plug-in connection for the culture vessel assembly on the socket 11. The construction of the culture vessel block 20 is described in more detail in our European patent application no. 03255896.7 and therefore will not be described further herein. However, it is important to note that the movable platen 23 is supported on the lid or platen 22 so as to be movable transversely relative to the lid (platen) 22 in order to cause agitation of the lower end of each stirring rod 25 within the culture vessel block wells 21.

The system 1 includes a drive rotor 14 which includes plural connectors 24, only one of which is shown (on the right hand side of the figure) which engage the platens 22,23. The connections are magnetic couplings (not shown) which engage with corresponding magnetic plate couplings 26,27 on the platens 22,23.

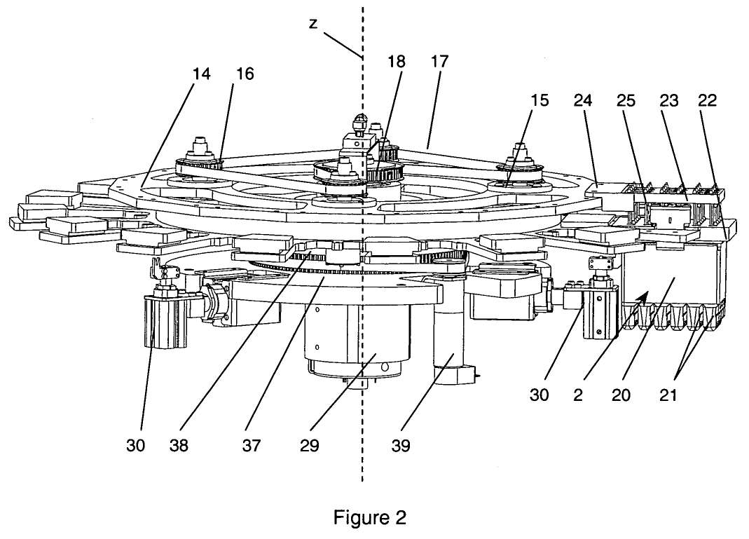

In order to move the stirring rods within the wells 21, it is necessary to move the platen 23 in an orbital fashion and this is achieved by mounting the rotor 14 for movement eccentrically with respect to the support 10. Four eccentric bearings 15 connect the rotor 14 to the support 10 (as best seen in FIG. 2) and each of the bearings 15 has an associated toothed pulley 16 which is engaged by a toothed drive belt 17 which is wound around the wheels 16 and driven by a toothed pulley wheel 18 connected to a first drive motor 29 (see FIG. 2). By operating the motor 29 selectively, the rotor 14 can be moved in an eccentric or orbital fashion relative to the support 10.

A second drive motor 39 (see FIG. 2) is also selectively operable, via a toothed belt 37 and a toothed pulley wheel 38 connected to the support 10, to rotate the support 10, when required, so that the culture vessel assemblies 2 can be loaded into or taken out of the support locations 11 as required. Loading of the culture vessel assemblies 2 is achieved by means of a robotic system (not shown) arranged, at a suitable position around the periphery of the support 10, to present a culture vessel assembly to a selected support location 11 and pushing the culture vessel assembly 2 into the support location. Unloading is achieved by first pushing the culture vessel assembly radially to break the magnetic couplings and then removing the culture vessel assembly 2 using the robotic system. The pushing system comprises a pair of pneumatic actuators 30, disposed at different positions around the axis z and pushing directly on a culture vessel assembly 2 in use.

In an alternative construction (not shown), the couplings may be non-magnetic. The system may also allow partial de-coupling, ie the platen 23 may be de-coupled from the connector 24 (and hence the drive rotor 14) while retaining the coupling of the platen 22 on the support location 11. This allows selected culture vessel assemblies to be partially removed during stirring of the contents of the remainder of the culture vessel assemblies which remain attached to the drive rotor 14.

In a further alternative construction (not shown), the support may not be rotatable itself, and loading/unloading of culture vessel assemblies may be achieved through a robotic system capable of moving around the support.

In an alternative construction, the stirring rods and platens 23 are fixed to the rotor 14 and engageable with the culture vessel blocks 20 when they are attached to the support 10.

1. A stirring system (1) for a cell culture system arranged to handle a plurality of cell culture vessel assemblies (2) each of which includes a platen (23) on which are disposed a plurality of stirring rods, and which is transversely movable across the top of the respective culture vessel to provide for stirring of one or more samples within the culture vessel, the stirring system including:
a support (10) configured to receive plural cell culture vessel assemblies at predefined positions around an axis (z);
a drive rotor (14) disposed adjacent the support, the drive rotor being mounted for eccentric movement about the axis (z) of the support (10) with respect to the support and having a plurality of drive connections (24) for selectively engaging respective platens (23) of culture vessel assemblies supported at the predefined positions to enable simultaneous stirring of samples in the plural culture vessels whose platens are engaged with the respective drive connections; and
a drive transmission system (15-19) for connecting the drive rotor to a drive motor (29).

2. A stirring system (1) for a cell culture system arranged to handle a plurality of cell culture vessel assemblies (2), the stirring system including:
a support (10) configured to receive plural cell culture vessel assemblies at a plurality of predefined positions around an axis (z);
a drive rotor (14) disposed adjacent the support, the drive rotor having a corresponding plurality of sets of stirring rods for disposing in use in respective culture vessel assemblies (2) supported at the predefined positions on the support (10), and being mounted for eccentric movement about the axis (z) of the support with respect to the support to vibrate the stirring rods to enable stirring of samples in the plural culture vessel assemblies (2) simultaneously; and
a drive transmission system (15-19) for connecting the drive rotor to a drive motor (29).

3. A system according to claim 1 or claim 2, wherein the support (10) is rotatable around the axis to move the cell culture vessel assemblies (2) between a number of positions about the axis (z).

4. A system according to claim 1 or claim 2, wherein the drive rotor (14) is disposed on the support (10).

5. A system according to claim 4, wherein the drive transmission system includes a plurality of eccentric bearings (15) disposed on one or both of the rotor (14) and the support (10).

6. A system according to claim 5, wherein the drive transmission system includes a drive pulley (18) mounted about the axis (z) and a drive belt (17) disposed around the drive pulley, and each of the bearings includes a pulley (16) around which the drive belt is disposed to drive the rotor (14).

7. A system according to claim 1, wherein each of the drive connections (24) includes a magnetic coupling engaging a corresponding magnetic coupling on a respective culture vessel assembly movable platen (23).

8. A system according to claim 1 or claim 2, wherein the culture vessel assemblies (2) are magnetically coupled to the support (10).

9. A system according to claim 1 or claim 2, wherein the culture vessel assemblies (2) are each coupled to the support (10) by means of a socket (11) on the support arranged to receive a corresponding plug-in portion of the culture vessel assembly.

10. A system according to claim 1 or claim 2, including one or more actuators disposed and actuatable to disengage culture vessel assemblies (2) from the support (10).

11. A system according to claim 1 or claim 2, including channels in the support for supplying gas or liquid to the culture vessel assemblies in use.

12. A system according to claim 11, wherein the stirring rods are tubular and are connected to the channels for supplying gas or liquid into the samples in the culture vessel assemblies.

13. A cell culture system including an incubator having a housing and, disposed within the housing a stirring system according to claim 1 or claim 2.

14. A cell culture system according to claim 13, wherein a plurality of stirring systems are disposed within the incubator housing one above another.
